(19) United States
(12) Patent Application Publication
(10) Pub. No.: US 2009/0071482 A1
(43) Pub. Date: Mar. 19, 2009

(54) Resuscitators

(75) Inventor: Mark Huddlestone, Jersey (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 11/919,946

(22) PCT Filed: May 5, 2006
(86) PCT No.: PCT/GB2006/001655

§ 371 Date: Nov. 6, 2007

(30) Foreign Application Priority Data
May 7, 2005 (GB) 0509371.1

(51) Int. Cl. A61M 16/00

(52) U.S. Cl. 128/205.13

(57) ABSTRACT

A manual squeeze bag resuscitator has an outlet (7) connected to a face mask or breathing tube via a length of flexible tubing (40) so that the resuscitator can be operated away from the face of the patient. A tube (10) within the bag (2) limits the extent by which the bag can be compressed in order to produce more repeatable operation. The tube (10) extends between the air inlet (6) and outlet (7) and has openings into the bag. The user may wear a wrist support (30), to limit flexing of the wrist, and the surface of this and the bag (2) may have a hook and loop fastener material to improve grip.

This invention relates to manual resuscitators of the kind including a resilient bag that can be squeezed to deliver gas to a patient and that recovers its original shape when released, the resuscitator including a valved inlet by which gas can enter the bag during recovery of its original shape and an outlet communicating with a coupling adapted to be connected to a patient face mask or breathing tube.

Manual resuscitators have been used successfully for many years. They have the advantage of being easy to use by relatively inexperienced users, they provide a positive feedback of ventilation pressure to the user and they are relatively low cost so that they can be provided for a single patient use. They do, however, suffer from some disadvantages. One major problem is that they can be tiring to use for prolonged periods, especially by users with small hands, which can lead to insufficient ventilation when the user becomes fatigued. Another problem is that the outlet of the resuscitator bag is connected directly to the face mask or breathing tube so the bag has to be supported in a raised position close to the patient's face. This also makes the resuscitator difficult to use for extended periods and obstructs the face region.

It is an object of the present invention to provide an alternative resuscitator.

According to one aspect of the present invention there is provided a manual resuscitator of the above-specified kind, characterised in that the outlet of the bag and the patient coupling are interconnected via a length of flexible tubing such that the bag can be located at a position away from the face mask or breathing tube.

The flexible tubing is preferably corrugated. The bag may include a member within it to limit the extent of compression of the wall of the bag.

According to another aspect of the present invention there is provided a manual resuscitator including a resilient bag that can be squeezed to deliver gas to a patient and that recovers its original shape when released, the resuscitator including a valved inlet by which gas can enter the bag during recovery of its original shape and an outlet communicating with a coupling adapted to be connected to a patient face mask or breathing tube, characterised in that the bag includes a member within it to limit the extent of compression of the wall of the bag.

The member within the bag is preferably a tube, which may be aligned between the inlet and outlet of the bag. The tube may open to the interior of the bag and the bore of the tube may communicate with the inlet and or alternatively the outlet of the bag. The tube may open to the interior of the bag via a plurality of holes in the tube. The resuscitator may include a wrist support adapted to be retained on the wrist to reduce flexing of the wrist when the bag is squeezed. The surface of the bag and the wrist support may be provided with cooperating hook and loop fastener materials.

A manual resuscitator will now be described, by way of example, with reference to the accompanying drawings, in which:

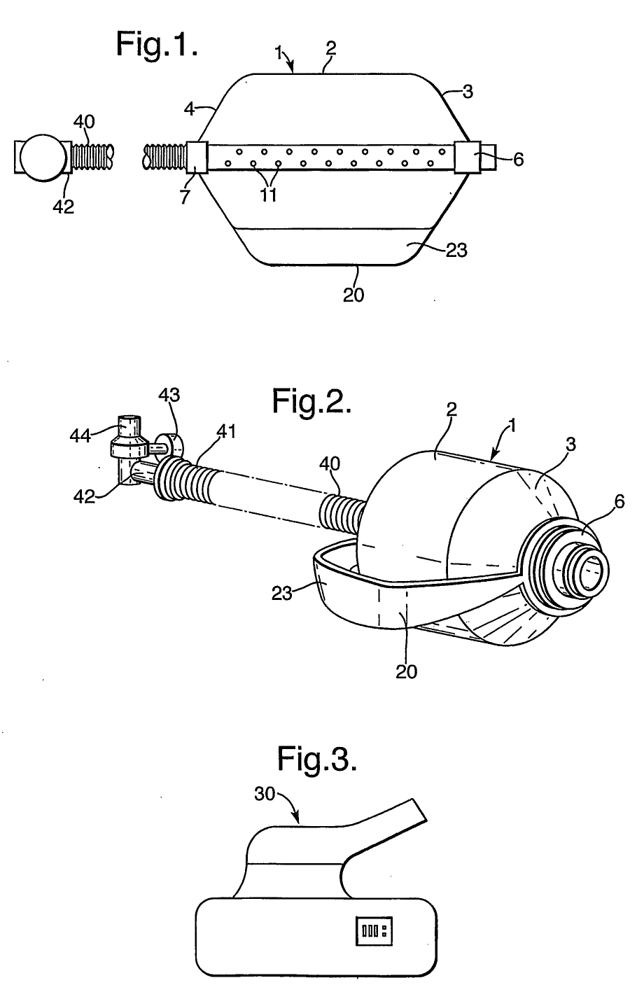

FIG. 1 is a cross-sectional side elevation view of the resuscitator;

FIG. 2 is a perspective view of the resuscitator;

FIG. 3 shows a wrist support; and

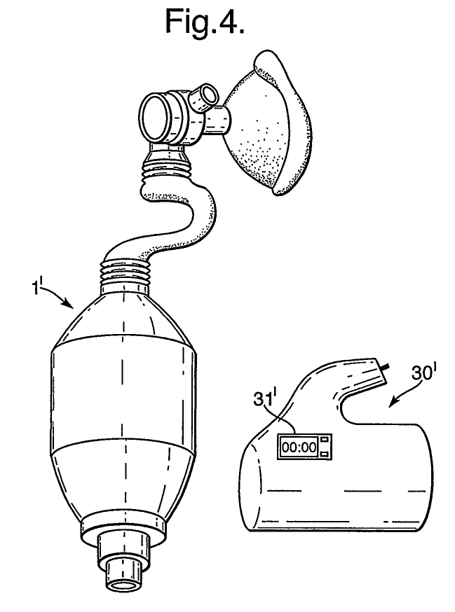

FIG. 4 is a perspective view of an alternative resuscitator.

With reference first to FIGS. 1 to 3, the resuscitator includes a squeeze bag 1 of drum shape and circular section, having a central portion 2 of cylindrical shape. The bag 1 has inlet and outlet portions 3 and 4 of a tapering shape. The wall 5 of the bag 1 is of a flexible plastics material, the thickness and shape being such that the bag is resilient, enabling it to be squeezed inwardly manually and, when released, it recovers its original shape. A rigid, valved inlet 6 is bonded to the wall 5 of the bag 1. This prevents air leaving the bag through the inlet when the bag is squeezed but allows air to enter the bag when the bag is released and recovers its original shape. The inlet 6 may include an oxygen inlet for use when the oxygen level needs to be supplemented above the level in ambient air. A rigid outlet 7 is bonded to the wall 5 of the bag at its outlet end and is unvalved. As so far described, the resuscitator is conventional.

The resuscitator differs from previous resuscitators by having means to limit the extent of compression of the wall of the bag. This takes the form of a hollow tube 10 extending between the inlet coupling 6 and the outlet coupling 7 axially of the bag. The tube 10 is formed with several opening 11 spaced along its length and around its circumference so that air can flow into and out of the tube freely and without risk of being blocked by contact with the wall 5 of the bag 1. The tube 10 limits how far the user can squeeze opposite sides of the bag 1 towards one another. This is believed to reduce fatigue in the hand and forearm that could otherwise be caused by prolonged operation. By limiting compression in this way, the volume of air delivered by each compression is more repeatable and constant.

There are other arrangements by which the extent of compression of the bag could be limited, instead of a tube within the bag On the outside of the bag 1 a plastics strap 20 extends in a loop having one end 21 attached to the inlet portion 3 and its other end 22 attached to the outlet portion 4. The strap 20 may be moulded integrally with the bag 1, of the same material. The strap is bendable but self-supporting so that, where it extends over the central portion 2 of the bag 1 it is spaced from the outside surface of the bag to form an opening 23 to receive the forward part of the user's hand. The hand is received as a loose fit in the opening 23 with the knuckle level with the strap 20 and the thumb extending away from the strap. The strap 20 helps support the bag 1 when it is being held in an elevated position and also helps stabilize the bag where it is compressed by pressing against a hard surface, such as the floor.

The resuscitator also includes a wrist support 30 arranged to be wrapped around the wrist and palm of the hand to stabilize the wrist and prevent excessive flexing. This is believed to help reduce fatigue both when the bag is squeezed between the fingers and thumb and when the bag is pressed on a hard surface.

The outlet coupling 7 of the bag is not connected directly to a face mask or breathing tube, as with conventional resuscitators but opens into a length of flexible corrugated tubing 40 fixed with the coupling. Typically, the tubing 40 would be significantly longer than the resuscitator bag itself. At the far end 41 of the tubing 40, remote from the bag 1, the tubing supports a conventional breathing valve 42 having a coupling 43 for connection to a face mask or breathing tube and an exhaust outlet 44. The valve 42 allows air to flow in one direction from the tubing 40 into the coupling 43 but diverts exhaled air flow from the coupling 43 to the exhaust outlet 44. The length of tubing 40 enables the user to operate the resuscitator bag 1 away from the face mask or breathing tube so that the bag can be held in the most convenient location for the user. It also enables the user to change the position of the bag 1 in order to reduce fatigue. Another advantage is that the tubing 40 enables the bag 1 to be squeezed by pressing against the floor or other hard surface. Because air exhaled by the patient is exhausted at the breathing valve 42, it does not enter the tubing 40. The tubing 40 does not, therefore, form a deadspace since it contains only fresh air, which will be administered to the patient.

In the alternative resuscitator shown in FIG. 4, the means for supporting the hand of the user is provided solely by a wrist support 30′, that is, the bag 1′ does not have a support strap. The wrist support 30′ includes an electronic display 31′ arranged to provide a display representation of the time for which resuscitation has been given. The display 31 may, therefore, take the form of a stop watch with a start button activated manually by the user. Alternatively, the display 31 could be linked to a sensor, such as a pressure sensor, so that the display starts automatically when the user starts to apply pressure to the bag 1′. In another embodiment, the display could provide a count, instead of a time, indication, such as in the manner of a pedometer, to indicate the number of ventilation cycles administered. The duration of ventilation could be indicated in a non-visual way, such as by an audible indication after a period of time or after a predetermined number of ventilation cycles.

In order to improve retention of the bag 1′ by the user wearing the wrist support 30′, the bag and wrist support could be provided with surfaces to improve grip. For example, the surface of the wrist support and bag could be provided with cooperating hook and loop fastener fabric material.

1. A manual resuscitator including a resilient bag that can be squeezed to deliver gas to a patient and that recovers its original shape when released, the resuscitator including a valved inlet by which gas can enter the bag during recovery of its original shape and an outlet communicating with a coupling adapted to be connected to a patient face mask or breathing tube, characterized in that the outlet of the bag and the patient coupling are interconnected via a length of flexible tubing such that the bag can be located at a position away from the face mask or breathing tube.

2. A manual resuscitator according to claim 1, characterized in that the flexible tubing is corrugated.

3. A manual resuscitator according to claim 1, characterized in that the bag includes a member within it to limit the extent of compression of the wall of the bag.

4. A manual resuscitator including a resilient bag that can be squeezed to deliver gas to a patient and that recovers its original shape when released, the resuscitator including a valved inlet by which gas can enter the bag during recovery of its original shape and an outlet communicating with a coupling adapted to be connected to a patient face mask or breathing tube, characterized in that the bag includes a member within it to limit the extent of compression of the wall of the bag.

5. A manual resuscitator according to claim 3, characterized in that the member within the bag is a tube.

6. A manual resuscitator according to claim 5, characterized in that the tube is aligned between the inlet and outlet of the bag.

7. A manual resuscitator according to claim 6, characterized in that the tube opens to the interior of the bag and that the bore of the tube communicates with the inlet and or alternatively the outlet of the bag.

8. A manual resuscitator according to claim 7, characterized in that the tube opens to the interior of the bag via a plurality of holes in the tube.

9. A manual resuscitator according to claim 1 including a wrist support adapted to be retained on the wrist to reduce flexing of the wrist when the bag is squeezed.

10. A manual resuscitator according to claim 9, characterized in that the surface of the bag and the wrist support are provided with cooperating hook and loop fastener materials.

11. A manual resuscitator according to claim 4, characterized in that the member within the bag is a tube.

12. A manual resuscitator according to claim 4, characterized in that the tube is aligned between the inlet and outlet of the bag.

13. A manual resuscitator according to claim 4, characterized in that the tube opens to the interior of the bag and that the bore of the tube communicates with the inlet and or alternatively the outlet of the bag.

14. A manual resuscitator according to claim 4 including a wrist support adapted to be retained on the wrist to reduce flexing of the wrist when the bag is squeezed.

15. A manual resuscitator according to claim 4 including a wrist support adapted to be retained on the wrist to reduce flexing of the wrist when the bag is squeezed.

16. A manual resuscitator according to claim 15, characterized in that the surface of the bag and the wrist support are provided with cooperating hook and loop fastener materials.
